SEQUENCE LISTING (20 sequences)

SEQ ID NO: 1 - protein, 112 aa, Mus sp.
  MISC_FEATURE: amino acid sequence of variable region of the heavy chain (VH) of murine monoclonal anti-CD20 antibody B-Ly1
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 2 - protein, 103 aa, Mus sp.
  MISC_FEATURE: amino acid sequence of variable region of the light chain (VL) of murine monoclonal anti-CD20 antibody B-Ly1
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 3 - protein, 119 aa, Artificial
  amino acid sequences of variable region of the heavy chain (VH) of humanized B-Ly1 antibody (B-HH2)
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 4 - protein, 119 aa, Artificial
  amino acid sequences of variable region of the heavy chain (VH) of humanized B-Ly1 antibody (B-HH3)
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 5 - protein, 119 aa, Artificial
  amino acid sequences of variable region of the heavy chain (VH) of humanized B-Ly1 antibody (B-HH4)
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 6 - protein, 119 aa, Artificial
  amino acid sequences of variable region of the heavy chain (VH) of humanized B-Ly1 antibody (B-HH5)
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 7 - protein, 119 aa, Artificial
  amino acid sequences of variable region of the heavy chain (VH) of humanized B-Ly1 antibody (B-HH6)
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 119 aa, Artificial
  amino acid sequences of variable region of the heavy chain (VH) of humanized B-Ly1 antibody (B-HH7)
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 9 - protein, 119 aa, Artificial
  amino acid sequences of variable region of the heavy chain (VH) of humanized B-Ly1 antibody (B-HH8)
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 10 - protein, 119 aa, Artificial
  amino acid sequences of variable region of the heavy chain (VH) of humanized B-Ly1 antibody (B-HH9)
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 11 - protein, 119 aa, Artificial
  amino acid sequences of variable region of the heavy chain (VH) of humanized B-Ly1 antibody (B-HL8)
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 12 - protein, 119 aa, Artificial
  amino acid sequences of variable region of the heavy chain (VH) of humanized B-Ly1 antibody (B-HL10)
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 13 - protein, 119 aa, Artificial
  amino acid sequences of variable region of the heavy chain (VH) of humanized B-Ly1 antibody (B-HL11)
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 14 - protein, 119 aa, Artificial
  amino acid sequences of variable region of the heavy chain (VH) of humanized B-Ly1 antibody (B-HL12)
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 15 - protein, 119 aa, Artificial
  amino acid sequences of variable region of the heavy chain (VH) of humanized B-Ly1 antibody (B-HL13)
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, Artificial
  amino acid sequences of variable region of the heavy chain (VH) of humanized B-Ly1 antibody (B-HL14)
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 119 aa, Artificial
  amino acid sequences of variable region of the heavy chain (VH) of humanized B-Ly1 antibody (B-HL15)
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 119 aa, Artificial
  amino acid sequences of variable region of the heavy chain (VH) of humanized B-Ly1 antibody (B-HL16)
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 119 aa, Artificial
  amino acid sequences of variable region of the heavy chain (VH) of humanized B-Ly1 antibody (B-HL17)
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 20 - protein, 115 aa, Artificial
  amino acid sequences of variable region of the light chain (VL) of humanized B-Ly1 antibody B-KV1
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

(19) United States
(12) Patent Application Publication
(10) Pub. No.: US 2013/0302274 A1
(43) Pub. Date: Nov. 14, 2013

(54) COMBINATION THERAPY

(71) Applicant: ROCHE GLYCART AG, Schlieren (CH)

(72) Inventors: CHRISTIAN KLEIN, BONSTETTEN (CH); EMILIE LAPREVOTTE, TOULOUSE (FR); ANNE QUILLET-MARY, TOULOUSE (FR)

(21) Appl. No.: 13/683,519

(22) Filed: Nov. 21, 2012

(30) Foreign Application Priority Data
Nov. 25, 2011 (FR) 1160787

(51) Int. Cl. A61K 39/395; A61K 45/06; A61K 38/20

(52) U.S. Cl. CPC A61K 39/39558; A61K 38/2033; A61K 45/06
USPC 424/85.2; 600/1

(57) ABSTRACT

The present invention is directed to the combination therapy of an afucosylated antibody specifically binding to a tumor-antigen with human IL-15 for the treatment of cancer.

### CROSS REFERENCE TO RELATED APPLICATIONS

This application claims benefit under 35 U.S.C. §119 to French Application No. 1160787, filed Nov. 25, 2011, which application is incorporated by reference herein.

### SEQUENCE LISTING

The instant application contains a Sequence Listing submitted via EFS-Web concurrently herewith and is hereby incorporated by reference in its entirety. Said ASCII copy, created on Nov. 13, 2012, is named P4840SeqList.txt, and is 24,270 bytes in size.

### FIELD OF THE INVENTION

The present invention is directed to the combination treatment of a patient suffering from cancer with an anti-CD20 antibody and the cytokine human IL-15, especially to the combination treatment of a patient suffering from hematological malignancies, such as leukemia, e.g. Chronic lymphocytic leukemia (CLL).

### BACKGROUND OF THE INVENTION

### Afucosylated Antibodies

Cell-mediated effector functions of monoclonal antibodies can be enhanced by engineering their oligosaccharide component as described in Umaña, P., et al., Nature Biotechnol. 17 (1999) 176-180; and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A. and Morrison, S. L., Trends Biotechnol. 15 (1997) 26-32). Umaña, P., et al., Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to Fc binding to FcγR and C1 q (Umaña, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Iida, S., et al., Clin. Cancer Res. 12 (2006) 2879-2887 show that efficacy of a afucosylated anti-CD20 antibody was inhibited by addition of fucosylated anti-CD20. The efficacy of a 1:9 mixture (10 microg/mL) of afucosylated and fucosylated anti-CD20s was inferior to that of a 1,000-fold dilution (0.01 microg/mL) of afucosylated anti-CD20 alone. They conclude that afucosylated IgG1, not including fucosylated counterparts, can evade the inhibitory effect of plasma IgG on ADCC through its high FcgammaRIIIa binding. Natsume, A., et al., shows in J. Immunol. Methods 306 (2005) 93-103 that fucose removal from complex-type oligosaccharide of human IgG1-type antibody results in a great enhancement of antibody-dependent cellular cytotoxicity (ADCC). Satoh, M., et al., Expert Opin. Biol. Ther. 6 (2006) 1161-1173 discusses afocusylated therapeutic antibodies as next-generation therapeutic antibodies. Satoh, M., concludes that antibodies consisting of only the afocusylated human IgG1 form is thought to be ideal. Kanda, Y., et al., Biotechnol. Bioeng. 94 (2006) 680-688 compared fucosylated CD20 antibody (96% fucosylation, CHO/DG44 1H5) with afocusylated CD20 antibody. Davies, J., et al., Biotechnol. Bioeng. 74 (2001) 288-294 reports that for a CD20 antibody increased ADCC correlates with increased binding to FcγRIII.

Methods to enhance cell-mediated effector functions of monoclonal antibodies by reducing the amount of fucose are described e.g. in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739, Niwa, R., et al., J. Immunol. Methods 306 (2005) 151-160; Shinkawa, T. et al, J Biol Chem, 278 (2003) 3466-3473; WO 03/055993 or US 2005/0249722.

### CD20 and Anti CD20 Antibodies

The CD20 molecule (also called human B-lymphocyte-restricted differentiation antigen or Bp35) is a hydrophobic transmembrane protein with a molecular weight of approximately 35 kD located on pre-B and mature B lymphocytes (Valentine, M. A., et al. J. Biol. Chem. 264(19) (1989) 11282-11287; and Einfield, D. A., et al. (1988) EMBO J. 7(3):711-717; Tedder, T. F., et al., Proc. Natl. Acad. Sci. U.S.A. 85 (1988) 208-12; Stamenkovic, I., et al., J. Exp. Med. 167 (1988) 1975-80; Tedder, T. F., et al., J. Immunol. 142 (1989) 2560-8). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson, K. C., et al., Blood 63(6) (1984) 1424-1433)) but is not found on hematopoietic stem cells, pro-B cells, normal plasma cells, or other normal tissues (Tedder, T. F., et al., J, Immunol. 135(2) (1985) 973-979).

The 85 amino acid carboxyl-terminal region of the CD20 protein is located within the cytoplasm. The length of this region contrasts with that of other B cell-specific surface structures such as IgM, IgD, and IgG heavy chains or histocompatibility antigens class I1 a or β chains, which have relatively short intracytoplasmic regions of 3, 3, 28, 15, and 16 amino acids, respectively (Komaromy, M., et al., NAR 11 (1983) 6775-6785). Of the last 61 carboxyl-terminal amino acids, 21 are acidic residues, whereas only 2 are basic, indicating that this region has a strong net negative charge. The GenBank Accession No. is NP-690605. It is thought that CD20 might be involved in regulating an early step(s) in the activation and differentiation process of B cells (Tedder, T. F., et al., Eur. J. Immunol. 16 (8) (1986) 881-887) and could function as a calcium ion channel (Tedder, T. F., et al., J. Cell. Biochem. 14D (1990) 195).

There exist two different types of anti-CD20 antibodies differing significantly in their mode of CD20 binding and biological activities (Cragg, M. S., et al, Blood, 103 (2004) 2738-2743; and Cragg, M. S., et al., Blood, 101 (2003) 1045-1052). Type I antibodies, as e.g. rituximab (a non-afocusylated, non-glycoengineered antibody with normal glycosylation pattern, also named “RTX”), are potent in complement mediated cytotoxicity, whereas type II antibodies, as e.g. Tositumomab (B1), 11B8, AT80 or humanized B-Ly1 antibodies, effectively initiate target cell death via caspase-independent apoptosis with concomitant phosphatidylserine exposure.

The sharing common features of type I and type II anti-CD20 antibodies are summarized in Table 1.

TABLE 1
Properties of type I and type II anti-CD20 antibodies

| type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross- | Strong cell death induction without |
| linking | cross-linking |

U.S. Pat. No. 5,736,137 relates to Rituximab which is a non-afocusylated, non-glycoengineered antibody with normal glycosylation pattern. WO 2005/044859 and WO 2007/031875 relate to afocusylated anti-CD20 antibodies with a reduced amount of fucose compared to the corresponding parent antibodies. WO 2008/121876 (A2,A3) relate to afocusylated anti-CD20 antibodies with a reduced amount of fucose compared to the corresponding parent antibodies.

### Cytokines:

### Properties of Cytokines

Cytokines are small secreted proteins which mediate and regulate immunity, inflammation, and hematopoiesis. They must be produced de novo in response to an immune stimulus. They generally (although not always) act over short distances and short time spans and at very low concentration. They act by binding to specific membrane receptors, which then signal the cell via second messengers, often tyrosine kinases, to alter its behavior (gene expression). Responses to cytokines include increasing or decreasing expression of membrane proteins (including cytokine receptors), proliferation, and secretion of effector molecules.

Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action).

It is common for different cell types to secrete the same cytokine or for a single cytokine to act on several different cell types (pleiotropy). Cytokines are redundant in their activity, meaning similar functions can be stimulated by different cytokines Cytokines are often produced in a cascade, as one cytokine stimulates its target cells to make additional cytokines. Cytokines can also act synergistically (two or more cytokines acting together) or antagonistically (cytokines causing opposing activities).

Their short half life, low plasma concentrations, pleiotropy, and redundancy all complicated the isolation and characterization of cytokines Searches for new cytokines is now often conducted at the DNA level, identifying genes similar to known cytokine genes.

### Cytokine Activities

Cytokine activities are characterized using recombinant cytokines and purified cell populations in vitro, or with knock-out mice for individual cytokine genes to characterize cytokine functions in vivo. Cytokines are made by many cell populations, but the predominant producers are helper T cells (Th) and macrophages.

Interleukin (IL)-15 belongs to a large cytokine family which includes IL-2, IL-4, IL-7, IL-9 and IL-21. Although these cytokines share the same gamma chain (γc) receptor, IL-2 and IL-15 have specific functions that are related both to their binding properties on the α-chains of the IL-2R and IL-15R as well as to their cellular activation mechanisms. The mechanism of IL-15 action is still under debate but seems to be trans-presentation by cellular partners such as monocytes and/or dendritic cells which are the main producers of IL-15 in vivo.

IL-15 displays important physiological functions facilitating innate and adaptative immunity; it has an important role in the development, homeostasis, and activation of immune cells such as Natural Killer (NK) or T lymphocytes cells. IL-15 is mostly trans-presented by accessory cells and has pleiotropic activities on NK cells: survival; proliferation; differentiation; increase in cytotoxic functions; stimulation of production of cytokines such as IFN-γ, TNF-α and GM-CSF; and regulation of NK/macrophage interactions. IL-15 also activates monocytes and macrophages leading to their involvement in anti-infectious immunity. In addition, IL-15 has been found to inhibit apoptosis of neutrophils and eosinophils as well as Fas-mediated apoptosis of B or T cells through up-regulation of anti-apoptotic proteins. A role for IL-15 in infectious or diseases has been reported.

### SUMMARY OF THE INVENTION

The invention comprises the use of an afucosylated antibody, preferably an antibody specifically binding to a tumor antigen with an amount of fucose of 60% or less, for the treatment of cancer in combination with the cytokines human IL-15.

In a preferred embodiment, the afucosylated antibody is an anti-CD20 antibody in combination with human IL-15 for use in treatment of cancer.

Preferably, the anti-CD20 antibody is characterized in that said CD20 antibody is an afucosylated antibody with an amount of fucose of 60% or less, and said cancer is a CD20 expressing cancer.

Preferably said anti-CD20 antibody is a humanized B-Ly1 antibody, and said cancer is a CD20 expressing cancer, preferably leukemia, more preferably Chronic Lymphocytic Leukemia (CLL).

One embodiment of the invention is characterized in that as cytokine only IL-15 is co-administered in said combination treatment.

One embodiment of the invention is characterized in that the afocusylated antibody shows an increased ADCC.

One embodiment of the invention is a composition comprising an anti-CD20 antibody and human IL-15 for the treatment of cancer.

The combination treatment of an anti-CD20 antibody in combination with the cytokine IL-15 shows enhanced antitumor inhibitory activity compared to a combination of the corresponding non-afocusylated, non-glycoengineered antibodies with the cytokine IL-15. The combination treatment mediates antitumor efficacy via transpresentation by accessory cells and is especially valuable for the treatment of cancers such as hematological malignancies such as CLL.

Preferably, the anti-CD20 antibody is characterized in that one or more additional other cytotoxic, chemotherapeutic or anti-cancer agents, or compounds or ionizing radiation that enhance the effects of such agents are administered.

Another embodiment of the invention relates to a method for the treatment of cancer, comprising administering to a patient in need of such treatment (i) an effective first amount of an anti-CD20 antibody; and (ii) an effective second amount of human IL-15.

### DETAILED DESCRIPTION OF THE INVENTION

The invention comprises the use of an afucosylated antibody of IgG1 or IgG3 isotype (preferably of IgG1 isotype) specifically binding to a tumor antigen with an amount of fucose of 60% or less of the total amount of oligosaccharides (sugars) at Asn297, for the manufacture of a medicament for the treatment of cancer in combination with the cytokines human IL-15, wherein the cancer expresses said tumor antigen.

In one embodiment the amount of fucose is between 20% and 60% of the total amount of oligosaccharides (sugars) at Asn297.

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained. The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. Human antibodies are well-known in the state of the art (van Dijk, M. A., and van de Winkel, J. G., Curr. Opin. Chem. Biol 5 (2001) 368-374). Based on such technology, human antibodies against a great variety of targets can be produced. Examples of human antibodies are for example described in Kellermann, S. A., et al., Curr Opin Biotechnol. 13 (2002) 593-597.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, the term “binding” or “specifically binding” refers to the binding of the antibody to an epitope of the tumor antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an afocusylated antibody according to the invention is specifically binding to the tumor antigen with a binding affinity (K_{D}) of 10⁻⁸ mol/l or less, preferably 10⁻⁹ M to 10⁻¹³ mol/1.

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC).

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a b-sheet conformation and the CDRs may form loops connecting the b-sheet structure. The CDRs in each chain are held in their three-dimensional structure by the framework regions and form together with the CDRs from the other chain the antigen binding site.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat, et al., Sequences of Proteins of Immunological Interest, 5th Ed. Public Health Service, National Institutes of Health, Bethesda, Md. (1991)) and/or those residues from a “hypervariable loop”.

The term “afucosylated antibody” refers to an antibody of IgG1 or IgG3 isotype (preferably of IgG1 isotype) with an altered pattern of glycosylation in the Fc region at Asn297 having a reduced level of fucose residues. Glycosylation of human IgG1 or IgG3 occurs at Asn297 as core fucosylated bianntennary complex oligosaccharide glycosylation terminated with up to 2 Gal residues. These structures are designated as G0, G1 (α1,6 or α1,3) or G2 glycan residues, depending from the amount of terminal Gal residues (Raju, T. S., BioProcess Int. 1 (2003) 44-53). CHO type glycosylation of antibody Fc parts is e.g. described by Routier, F. H., Glycoconjugate J. 14 (1997) 201-207. Antibodies which are recombinantely expressed in non glycomodified CHO host cells usually are fucosylated at Asn297 in an amount of at least 85%.

Thus an afucosylated antibody according to the invention means an antibody of IgG1 or IgG3 isotype (preferably of IgG1 isotype) wherein the amount of fucose is 60% or less of the total amount of oligosaccharides (sugars) at Asn297 (which means that at least 40% or more of the oligosaccharides of the Fc region at Asn297 are afucosylated). In one embodiment the amount of fucose is between 20% and 60% of the oligosaccharides of the Fc region at Asn297. In one embodiment the amount of fucose is between 40% and 60% of the oligosaccharides of the Fc region at Asn297. In another embodiment the amount of fucose is 50% or less, and in still another embodiment the amount of fucose is 30% or less of the oligosaccharides of the Fc region at Asn297. According to the invention “amount of fucose” means the amount of said oligosaccharide (fucose) within the oligosaccharide (sugar) chain at Asn297, related to the sum of all oligosaccharides (sugars) attached to Asn 297 (e.g. complex, hybrid and high mannose structures) measured by MALDI-TOF mass spectrometry and calculated as average value (for a detailed procedure to determine the amount of fucose, see Example 8).

Furthermore the oligosaccharides of the Fc region are preferably bisected. The afucosylated antibody according to the invention can be expressed in a glycomodified host cell engineered to express at least one nucleic acid encoding a polypeptide having GnTIII activity in an amount sufficient to partially fucosylate the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions (e.g. fermentation time) or by combination of at least two antibodies with different fucosylation amount. Such afucosylated antibodies and respective glycoengineering methods are described in WO 2005/044859, WO 2004/065540, WO2007/031875, Umana, P., et al., Nature Biotechnol. 17 (1999) 176-180, WO 99/154342, WO 2005/018572, WO 2006/116260, WO 2006/114700, WO 2005/011735, WO 2005/027966, WO 97/028267, US 2006/0134709, US 2005/0054048, US 2005/0152894, WO 2003/035835, WO 2000/061739. These glycoengineered antibodies have an increased ADCC. Other glycoengineering methods yielding afocusylated antibodies according to the invention are described e.g. in Niwa, R., et al., J. Immunol. Methods 306 (2005) 151-160; Shinkawa, T. et al, J Biol Chem, 278 (2003) 3466-3473; WO 03/055993 or US 2005/0249722.

One embodiment of the invention is characterized in that the afocusylated antibody shows an increased ADCC (compared to the corresponding non-afocusylated parent antibody). In one embodiment the afocusylated antibody has an increased ADCC compared to the corresponding non-afocusylated parent antibody of at least 50% (at 10 ng/ml antibody concentration and a effector cells/tumor cell E:T ratio of 25:1 with freshly isolated PBMC as Effector cells and suitable antigen-expressing tumor cells (e.g. Raji for CD20).

The afucosylated antibodies according to the invention, as e.g. anti-CD20 antibodies, have an increased antibody dependent cellular cytotoxicity (ADCC).

By “afucosylated antibodies (e.g. anti-CD20 antibodies) with increased antibody dependent cellular cytotoxicity (ADCC)” is meant an afucosylated antibody (e.g. anti-CD20 antibody), as that term is defined herein, having increased ADCC as determined by any suitable method known to those of ordinary skill in the art.

One accepted in vitro ADCC assay to determine the increased ADCC of the afocusylated antibody compared to the corresponding wild type parent antibody is described in WO 2005/044859:

1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
i) the PBMCs are isolated using standard density centrifugation procedures and are suspended at 5×10⁶ cells/ml in RPMI cell culture medium;
ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
iii) 100 microliters of the final target cell suspension above are transferred to each well of a 96-well microtiter plate;
iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
vi) for the spontaneous release (SR) controls, 3 additional wells in the plate containing the labeled target cells, receive 50 microliters of RPMI cell culture medium instead of the antibody solution (point iv above);
vii) the 96-well microtiter plate is then centrifuged at 50×g for 1 minute and incubated for 1 hour at 4° C.;
viii) 50 microliters of the PBMC suspension (point i above) are added to each well to yield an effector:target cell ratio of 25:1 and the plates are placed in an incubator under 5% CO2 atmosphere at 37 C for 4 hours;
ix) the cell-free supernatant from each well is harvested and the experimentally released radioactivity (ER) is quantified using a gamma counter;
x) the percentage of specific lysis is calculated for each antibody concentration according to the formula (ER−MR)/(MR−SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In a preferred embodiment increased ADCC is defined as an increase in the percentage of specific lysis observed at 10 ng/ml antibody concentration and a effector cells/tumor cell E:T ratio of 25:1 with freshly isolated PBMC as Effector cells and suitable antigen-expressing tumor cells (e.g. Raji for CD20 after 4 h).

The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

Said “increased ADCC” can be obtained by glycoengineering of said antibodies, that means enhance said natural, cell-mediated effector functions of monoclonal antibodies by engineering their oligosaccharide component as described in Umana, P., et al., Nature Biotechnol. 17 (1999) 176-180 and U.S. Pat. No. 6,602,684. The amount of fucose in such glycoengineered antibodies is 60% or lower, whereas the amount of fucose int eh correspongin wild type parent antibodies (in which the glycostructure is not engineered) is usually 85% or higher.

The term “complement-dependent cytotoxicity (CDC)” refers to lysis of human tumor target cells by the antibody according to the invention in the presence of complement. CDC is measured preferably by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. The assay is performed preferably with ⁵¹Cr or Eu labeled tumor cells and measurement of released ⁵¹Cr or Eu. Controls include the incubation of the tumor target cells with complement but without the antibody.

A “tumor antigen,” as used herein, refers to a tumor antigen of human origin and includes the meaning known in the art, which includes any molecule expressed on (or associated with the development of) a tumor cell that is known or thought to contribute to a tumorigenic characteristic of the tumor cell. Numerous tumor antigens are known in the art. Whether a molecule is a tumor antigen can also be determined according to techniques and assays well known to those skilled in the art, such as for example clonogenic assays, transformation assays, in vitro or in vivo tumor formation assays, gel migration assays, gene knockout analysis, etc. Preferably the term “tumor antigen” when used herein refers to a human transmembrane protein i.e., a cell membrane proteins which is anchored in the lipid bilayer of cells. The human transmembrane protein will generally comprise an “extracellular domain” as used herein, which may bind a ligand; a lipophilic transmembrane domain, a conserved intracellular domain tyrosine kinase domain, and a carboxyl-terminal signaling domain harboring several tyrosine residues which can be phosphorylated. The tumor antigen include molecules such as EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably MCSP, EGFR, CEA, CD20, or IGF1-R, more preferably CD20. Therefore said afucosylated antibody according to the invention is preferably an anti-CD20 antibody.

Thus one aspect of the invention is the use of an afucosylated antibody of IgG1 or IgG3 isotype (preferably of IgG1 isotype) specifically binding to a tumor antigen with an amount of fucose of 60% or less of the total amount of oligosaccharides (sugars) at Asn297, for the manufacture of a medicament for the treatment of cancer in combination with one or more cytokines selected from the group of GM-CSF, M-CSF and IL-15, wherein the tumor antigen is selected from EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably from MCSP, EGFR, CEA, CD20, or IGF1-R, more preferably CD20. In one embodiment the amount of fucose is between 20% and 60% of the total amount of oligosaccharides (sugars) at Asn297. In another embodiment the amount of fucose is between 40% and 60% of the total amount of oligosaccharides (sugars) at Asn297.

Thus one aspect of the invention is the use of an afucosylated antibody of IgG1 or IgG3 isotype (preferably of IgG1 isotype) specifically binding to a tumor antigen with an amount of fucose of 60% or less of the total amount of oligosaccharides (sugars) at Asn297, for the manufacture of a medicament for the treatment of cancer in combination with one or more cytokines selected from the group of GM-CSF, M-CSF and IL-15, wherein the cancer expresses said tumor antigen which is selected from EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably from MCSP, EGFR, CEA, CD20, or IGF1-R, more preferably CD20. In one embodiment the amount of fucose is between 20% and 60% of the total amount of oligosaccharides (sugars) at Asn297. In another embodiment the amount of fucose is between 40% and 60% of the total amount of oligosaccharides (sugars) at Asn297.

As used herein, the term “binding” or “specifically binding” refers to the binding of the antibody to an epitope of the antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an afocusalyted antibody according to the invention is specifically binding to a tumor antigen for which it is specific with a binding affinity (K_{D}) of 10⁻⁸ mol/l or less, preferably 10⁻⁹ M to 10⁻¹³ mol/1.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A., et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC.

Synonyms of CD20, as recognized in the art, include B-lymphocyte antigen CD20, B-lymphocyte surface antigen B1, Leu-16, Bp35, BM5, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table 2.

TABLE 2
Properties of type I and type II anti-CD20 antibodies

| Type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross- | Strong cell death induction without |
| linking | cross-linking |

Examples of type II anti-CD20 antibodies include e.g. humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype.

Examples of type I anti-CD20 antibodies include e.g. rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

The afucosylated anti-CD20 antibodies according to the invention is preferably a type II anti-CD20 antibodies, more preferably an afucosylated humanized B-Ly1 antibody as described in WO 2005/044859 and WO 2007/031875.

The “rituximab” antibody (reference antibody; example of a type I anti-CD20 antibody) is a genetically engineered chimeric human gamma 1 murine constant domain containing monoclonal antibody directed against the human CD20 antigen. However this antibody is not glycoengineered and not afocusylates and thus has an amount of fucose of at least 85%. This chimeric antibody contains human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC). The term “humanized B-Ly1 antibody” refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 1; variable region of the murine light chain (VL): SEQ ID NO: 2-see Poppema, S, and Visser, L., Biotest Bulletin 3 (1987) 131-139) by chimerization with a human constant domain from IgG1 and following humanization (see WO 2005/044859 and WO 2007/031875). These “humanized B-Ly1 antibodies” are disclosed in detail in WO 2005/044859 and WO 2007/031875.

Preferably the “humanized B-Ly1 antibody” has variable region of the heavy chain (VH) selected from group of SEQ ID No. 3 to SEQ ID No. 19 (B-HH2 to B-HH9 and B-HL8 to B-HL17 of WO 2005/044859 and WO 2007/031875). Especially preferred are Seq. ID No. 3, 4, 7, 9, 11, 13 and 15 (B-HH2, BHH-3, B-HH6, B-HH8, B-HL8, B-HL11 and B-HL13 of WO 2005/044859 and WO 2007/031875). Preferably the “humanized B-Ly1 antibody” has variable region of the light chain (VL) of SEQ ID No. 20 (B-KV1 of WO 2005/044859 and WO 2007/031875). Preferably the “humanized B-Ly1 antibody” has a variable region of the heavy chain (VH) of SEQ ID No. 7 (B-HH6 of WO 2005/044859 and WO 2007/031875) and a variable region of the light chain (VL) of SEQ ID No. 20 (B-KV1 of WO 2005/044859 and WO 2007/031875). This humanized B-Ly1 antibody as used herein is named “GA101” or “obinutuzumab” (WHO Drug Information, Vol. 25, No. 1, 2011). Said antibody is preferred. Furthermore the humanized B-Ly1 antibody is preferably an IgG1 antibody. According to the invention such afocusylated humanized B-Ly1 antibodies are glycoengineered (GE) in the Fc region according to the procedures described in WO 2005/044859, WO 2004/065540, WO2007/031875, Umana, P., et al., Nature Biotechnol. 17 (1999) 176-180 and WO 99/154342. The afucosylated glyco-engineered humanized B-Ly1 (B-HH6-B-KV1 GE) is preferred in one embodiment of the invention. Such glycoengineered humanized B-Ly1 antibodies have an altered pattern of glycosylation in the Fc region, preferably having a reduced level of fucose residues. Preferably the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). Furthermore the oligosaccharides of the Fc region are preferably bisected. These glycoengineered humanized B-Ly1 antibodies have an increased ADCC.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. (Jenkins, N., et al., Nature Biotechnol. 14 (1996) 975-81).

Mammalian cells are the preferred hosts for production of therapeutic glycoproteins, due to their capability to glycosylate proteins in the most compatible form for human application. (Cumming, D. A., et al., Glycobiology 1 (1991) 115-30; Jenkins, N., et al., Nature Biotechnol. 14 (1996) 975-81). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum free media, and permit the development of safe and reproducible bioprocesses. Other commonly used animal cells include baby hamster kidney (BHK) cells, NSO- and SP2/0-mouse myeloma cells. More recently, production from transgenic animals has also been tested. (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion or functional activity. (Wright, A., and Morrison, S. L., Trends Biotech. 15 (1997) 26-32). The structure of the attached N-linked carbohydrate varies considerably, depending on the degree of processing, and can include high-mannose, multiply-branched as well as biantennary complex oligosaccharides. (Wright, A., and Morrison, S. L., Trends Biotech. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R., et al., Glycobiology 5(8) (1995) 813-22).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umana, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32).

It was previously shown that overexpression in Chinese hamster ovary (CHO) cells of β(1,4)—N-acetylglucosaminyltransferase I11 (“GnTII17y), a glycosyltransferase catalyzing the formation of bisected oligosaccharides, significantly increases the in vitro ADCC activity of an antineuroblastoma chimeric monoclonal antibody (chCE7) produced by the engineered CHO cells. (See Umana, P., et al., Nature Biotechnol. 17 (1999) 176-180; and WO 99/154342, the entire contents of which are hereby incorporated by reference). The antibody chCE7 belongs to a large class of unconjugated monoclonal antibodies which have high tumor affinity and specificity, but have too little potency to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, above the levels found in naturally-occurring antibodies.

Interleukin (IL)-15 belongs to a large cytokine family which includes IL-2, IL-4, IL-7, IL-9 and IL-21. Although these cytokines share the same gamma chain (γc) receptor, 1 IL-2 and IL-15 have specific functions that are related both to their binding properties on the α-chains of the IL-2R and IL-15R 2 as well as to their cellular activation mechanisms. Recombinant Human IL-15 is commercially available, e.g. from Peprotech (Tebu-bio, Le Perray-en-Yvelines, France).

The term “cancer” as used herein refers to cancers or tumors which express the tumor antigen to which the afocusylated antibody is specifically binding. Such cancers includes lymphomas, lymphocytic leukemias, preferably acute or chronic lymphocytic leukemia, myeloid leukemia, preferably acute or chronic myeloid leukemia, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers. Preferably, said cancer is chronic lymphocytic leukemia (CLL).

Preferably the combination treatment of an afocusylated antibody according to the invention in combination with a cytokine selected from human GM-CSF, human M-CSF and/or human IL3 (which all differentiate human monocytes/pericytes into macrophage) is used for the treatment of cancers or tumors which are infiltrated by monocytes/pericytes; and is especially valuable for treatment of cancers or tumors with a high infiltration by monocytes/pericytes. The monocytes/pericytes-infiltration of cancers or tumors can be detected (in the tumor tissue after biopsy) by monocytes/pericyte-specific staining using monocyte-specific markers like CD14 (Wright S. D. et al., Science 249 (1990) 1431-1433; Bogman M. J. et al., Transplant Proc. 23 (1991) 1293-1294; Andreesen, R, et al., J Leukoc Biol. 47(6) (1990) 490-7). Typically, a person skilled in the art will use the combination treatment of an afocusylated antibody according to the invention in combination with a cytokine selected from human GM-CSF, human M-CSF and/or human IL3 for the treatment of monocytes/pericytes-infiltrated cancers or tumors which express the tumor antigen to which the afocusylated antibody is specifically binding. So in one embodiment the cancer is a monocytes/pericytes-infiltrated cancer (detectable by the monocyte specific CD14 antigen).

The term “expression of the CD20” antigen is intended to indicate an significant level of expression of the CD20 antigen in a cell, preferably on the cell surface of a T- or B-Cell, more preferably a B-cell, from a tumor or cancer, respectively, preferably a non-solid tumor. Patients having a “CD20 expressing cancer” can be determined by standard assays known in the art. E.g. CD20 antigen expression is measured using immunohistochemical (IHC) detection, FACS or via PCR-based detection of the corresponding mRNA.

The term “CD20 expressing cancer” as used herein refers to all cancers in which the cancer cells show an expression of the CD20 antigen. Preferably CD20 expressing cancer as used herein refers to lymphomas (preferably B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma) c) marginal zone lymphomas (including extranodal marginal zone B cell lymphoma (Mucosa-associated lymphatic tissue lymphomas, MALT), nodal marginal zone B cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma) f) hairy cell leukemia, g) lymphocytic lymphoma, waldenstrom's macroglobulinemia, h) acute lymphocytic leukemia (ALL), chronic lymphocytic leukemia (CLL)/small lymphocytic lymphoma (SLL), B-cell prolymphocytic leukemia, i) plasma cell neoplasms, plasma cell myeloma, multiple myeloma, plasmacytoma j) Hodgkin's disease.

More preferably the CD20 expressing cancer is a B-Cell Non-Hodgkin's lymphomas (NHL). Especially the CD20 expressing cancer is a Mantle cell lymphoma (MCL), acute lymphocytic leukemia (ALL), chronic lymphocytic leukemia (CLL), B-cell diffuse large cell lymphoma (DLCL), Burkitt's lymphoma, hairy cell leukemia, follicular lymphoma, multiple myeloma, marginal zone lymphoma, post transplant lymphoproliferative disorder (PTLD), HIV associated lymphoma, waldenstrom's macroglobulinemia, or primary CNS lymphoma.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “co-administration” or “co-administering” refer to the administration of said afucosylated antibody, preferably the afucosylated anti-CD20 antibody), and human IL-15 as one single formulation or as two separate formulations. The co-administration can be simultaneous or sequential in either order, wherein preferably there is a time period while both (or all) active agents simultaneously exert their biological activities. Said afucosylated antibody and human IL-15 are co-administered either simultaneously or sequentially (e.g. via an intravenous (i.v.) through a continuous infusion (one for the antibody and eventually one for the human IL-15). When both therapeutic agents are co-administered sequentially the dose is administered either on the same day in two separate administrations, or one of the agents is administered on day 1 and the second is co-administered on day 2 to day 7, preferably on day 2 to 4. Thus the term “sequentially” means within 7 days after the dose of the first component (cytokine or antibody), preferably within 4 days after the dose of the first component; and the term “simultaneously” means at the same time. The terms “co-administration” with respect to the maintenance doses of said afucosylated antibody and the human IL-15 mean that the maintenance doses can be either co-administered simultaneously, if the treatment cycle is appropriate for both drugs, e.g. every week. Or human IL-15 is e.g. administered e.g. every first to third day and said afucosylated antibody is administered every week. Or the maintenance doses are co-administered sequentially, either within one or within several days.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration of said afucosylated antibody and human IL-15 and the timing of co-administration will depend on the type (species, gender, age, weight, etc.) and condition of the patient being treated and the severity of the disease or condition being treated. Said afucosylated antibody and human IL-15 are suitably co-administered to the patient at one time or over a series of treatments. Depending on the type and severity of the disease, about 1 μg/kg to 50 mg/kg (e.g. 0.1-20 mg/kg) of said afucosylated antibody and 1 μg/kg to 50 mg/kg (e.g. 0.1-20 mg/kg) of human IL-15 is an initial candidate dosage for co-administration of both drugs to the patient. If the administration is intravenous the initial infusion time for said afucosylated antibody or human IL-15 may be longer than subsequent infusion times, for instance approximately 90 minutes for the initial infusion, and approximately 30 minutes for subsequent infusions (if the initial infusion is well tolerated).

The preferred dosage of said afucosylated antibody will be in the range from about 0.1 mg/kg to about 50 mg/kg. Thus, one or more doses of about 0.5 mg/kg, 2.0 mg/kg, 4.0 mg/kg, 10 mg/kg or 30 mg/kg (or any combination thereof) may be co-administered to the patient. In one embodiment the preferred dosage of said afucosylated anti-CD20 antibody (preferably the afocusylated humanized B-Ly1 antibody) will be in the range from about 0.05 mg/kg to about 30 mg/kg. Thus, one or more doses of about 0.5 mg/kg, 2.0 mg/kg, 4.0 mg/kg, 10 mg/kg or 30 mg/kg (or any combination thereof) may be co-administered to the patient. The preferred dosage of human IL-15 will be in the range from 0.01 mg/kg to about 50 mg/kg, e.g. 0.1 mg/kg to 10.0 mg/kg for human IL-15. Depending on the on the type (species, gender, age, weight, etc.) and condition of the patient and on the type of afucosylated antibody and human IL-15, the dosage and the administration schedule of said afucosylated antibody can differ from the dosage of human IL-15. E.g. the said afucosylated antibody may be administered e.g. every one to three weeks and human IL-15 may be administered daily or every 2 to 10 days. An initial higher loading dose, followed by one or more lower doses may also be administered.

In one embodiment the preferred dosage of said afucosylated anti-CD20 antibody (preferably the afocusylated humanized B-Ly1 antibody) will be 800 to 1600 mg (in on embodiment 800 to 1200 mg) on day 1, 8, 15 of a 3- to 6-weeks-dosage-cycle and then in a dosage of 400 to 1200 (in one embodiment 800 to 1200 mg on day 1 of up to nine 3- to 4-weeks-dosage-cycles.

In a preferred embodiment, the medicament is useful for preventing or reducing metastasis or further dissemination in such a patient suffering from cancer, preferably from monocytes/pericytes infiltrated cancers. The medicament is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, the medicament is useful for increasing the response rate in a group of patients.

In the context of this invention, additional other cytotoxic, chemotherapeutic or anti-cancer agents, or compounds that enhance the effects of such agents (e.g. cytokines) may be used in the afucosylated antibody and human IL-15 combination treatment of cancer. Such molecules are suitably present in combination in amounts that are effective for the purpose intended. Preferably the said afucosylated antibody human IL-15 combination treatment is used without such additional cytotoxic, chemotherapeutic or anti-cancer agents, or compounds that enhance the effects of such agents.

Such agents include, for example: alkylating agents or agents with an alkylating action, such as cyclophosphamide (CTX; e.g. Cytoxan®), chlorambucil (CHL; e.g. Leukeran®), cisplatin (Cis P; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil. Preferably the afucosylated antibody and IL-15 combination treatment is used without such additional agents.

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used in addition to the afucosylated antibody and human IL-15 combination treatment of CD20 expressing cancer. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Is also possible to label the antibody with such radioactive isotopes. Preferably the afucosylated antibody and human IL-15 combination treatment is used without such ionizing radiation.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023.

The afucosylated antibodies are administered to a patient according to known methods, by intravenous administration as a bolus or by continuous infusion over a period of time, by intramuscular, intraperitoneal, intracerobrospinal, subcutaneous, intra-articular, intrasynovial, or intrathecal routes. Intravenous or subcutaneous administration of the antibodies is preferred.

The human IL-15 is administered to a patient according to known methods, e.g. by intravenous administration as a bolus or by continuous infusion over a period of time, by intramuscular, intraperitoneal, intracerobrospinal, subcutaneous, intra-articular, intrasynovial, intrathecal, or peroral routes. Intravenous, subcutaneous or oral administration of human IL-15 is preferred.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

### Pharmaceutical Compositions

Pharmaceutical compositions can be obtained by processing the afucosylated antibodies according to the invention, as e.g. the anti-CD20 antibodies, and human IL-15 according to this invention with pharmaceutically acceptable, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

One embodiment of the invention is composition comprising both said afucosylated antibody with an amount of fucose is 60% or less (preferably said afucosylated anti-CD20 antibody) and human IL-15, for use in the treatment of cancer, in particular of CD20 expressing cancer.

Said pharmaceutical composition may further comprise one or more pharmaceutically acceptable carriers.

The present invention further provides a pharmaceutical composition, in particular for use in cancer, comprising (i) an effective first amount of an afucosylated antibody with an amount of fucose is 60% or less (preferably an afucosylated anti-CD20 antibody), and (ii) an effective second amount of human IL-15. Such composition optionally comprises pharmaceutically acceptable carriers and/or excipients.

Pharmaceutical compositions of the afucosylated antibody alone used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the human IL-15 depend on their pharmaceutical properties. Such compositions can be similar to those describe above for the afucosylated antibody.

In one further embodiment of the invention the pharmaceutical compositions according to the invention are preferably two separate formulations for said afucosylated antibody and human IL-15.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The present invention further provides a method for the treatment of cancer, comprising administering to a patient in need of such treatment (i) an effective first amount of an afucosylated antibody with an amount of fucose is 60% or less, (preferably an afucosylated anti-CD20 antibody); and (ii) an effective second amount of human IL-15.

In one embodiment the method is characterized in that the afocusylated antibody shows an increased ADCC.

In one embodiment the method is characterized in that said afucosylated antibody is an anti-CD20 antibody and said cancer is a CD20 expressing cancer.

In one embodiment the method is characterized in that said afucosylated anti-CD20 antibody is a humanized B-Ly1 antibody.

In one embodiment the method is characterized in that said afucosylated anti-CD20 antibody is obinutuzumab.

In one embodiment the method is characterized in that said the cancer is a monocytes/pericytes-infiltrated cancer.

In one embodiment the method is characterized in that as cytokine only IL-15 is co-administered in said combination treatment.

In one embodiment the method is characterized in that one or more additional other cytotoxic, chemotherapeutic or anti-cancer agents, or compounds or ionizing radiation that enhance the effects of such agents are administered.

As used herein, the term “patient” preferably refers to a human in need of treatment with an afucosylated antibody, preferably an afucosylated anti-CD20 antibody) (e.g. a patient suffering from CD20 expressing cancer, respectively) for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others.

The invention further comprises an afucosylated antibody, preferably an afucosylated anti-CD20 antibody, for the treatment of cancer in combination with human IL-15.

The invention further comprises an afucosylated antibody specifically binding to a tumor antigen (which is CD20) with an amount of fucose is 60% or less, and human IL-15 for the treatment of cancer.

In one embodiment that said the cancer is a monocytes/pericytes-infiltrated cancer.

In one embodiment the afocusylated antibody shows an increased ADCC.

In one embodiment said afucosylated antibody is an anti-CD20 antibody and said cancer is a CD20 expressing cancer.

In one embodiment said afucosylated anti-CD20 antibody is a humanized B-Ly1 antibody.

In one embodiment the afucosylated antibody (preferably the afucosylated anti-CD20 antibody) is used in combination with IL-15.

Preferably the CD20 expressing cancer is a B-Cell Non-Hodgkin's lymphoma (NHL). More preferable, the CD20 expressing cancer is Chronic Lymphocytic Leukemia (CLL).

The following examples and figures are provided to aid the understanding of the present invention, the true scope of which is set forth in the appended claims. It is understood that modifications can be made in the procedures set forth without departing from the spirit of the invention.

### SEQUENCE LISTING INFORMATION

SEQ ID NO: 1 amino acid sequence of variable region of the heavy chain (VH) of murine monoclonal anti-CD20 antibody B-Ly1.
SEQ ID NO: 2 amino acid sequence of variable region of the light chain (VL) of murine monoclonal anti-CD20 antibody B-Ly1.
SEQ ID NO: 3-19 amino acid sequences of variable region of the heavy chain (VH) of humanized B-Ly1 antibodies (B-HH2 to B-HH9, B-HL8, and B-HL10 to B-HL17)
SEQ ID NO: 20 amino acid sequences of variable region of the light chain (VL) of humanized B-Ly1 antibody B-KV1

### DESCRIPTION OF THE FIGURES

FIG. 1: NK cell activation induced by monoclonal antibodies and IL-15 in CLL samples. PBMC from CLL patients were treated or not with RTX (10 μg/mL) or GA101 (10 μg/mL) with or without IL-15 (10 ng/mL) for 7 days then analyzed by flow cytometry. Results represent the percentage of CD69⁺ NK cells among total NK cells after 7 days' culture (n=26) in the presence or not of monoclonal antibodies and IL-15. Horizontal bars represent the median value (**p<0.01 and ***p<0.001).

FIG. 2: NK cell proliferation induced by monoclonal antibodies and IL-15 in CLL samples. PBMC from CLL patients were treated or not with RTX (10 μg/mL) or GA101 (10 μg/mL) with or without IL-15 (10 ng/mL) for 7 days then analyzed by flow cytometry. (A) Representative experiment of CFSE dilution on NK cells in the presence or not of monoclonal antibodies and IL-15 (n=19). (B) Percentage of NK cells under proliferation among total NK cells after 7 days' culture in the presence or not of anti-CD20 monoclonal antibodies (n=17) and IL-15. Horizontal bars represent the median value (**p<0.01 and ***p<0.001).

FIG. 3: Proliferation of purified NK cells from healthy donors by B leukemic cell supernatants and monoclonal antibodies with or without IL-15. Results show a representative experiment of CFSE dilution on NK cells in the presence or not of RTX (10 μg/mL) or GA101 (10 μg/mL) for 7 days then analyzed by flow cytometry (n=5). (A) CFSE-labelled purified NK cells from healthy donors were cultured in culture medium with or without IL-15 (10 ng/mL). (B) Purified B leukemic cells were stimulated or not with IL-15 for 7 days. Supernatants were used as culture medium for analysis of CFSE-labelled purified NK cell proliferation.

FIG. 4: NK cell proliferation induced by monoclonal antibodies and IL-15 in total samples versus monocyte- or monocyte/CD3⁺-depleted CLL samples. Total, monocyte- or monocyte/CD3⁺-depleted samples from CLL patients were treated or not with RTX (10 μg/mL), or GA101 (10 μg/mL) with or without IL-15 (10 ng/mL) for 7 days then analyzed by flow cytometry. (A) Representative experiment of CFSE dilution on NK cells in the presence or not of monoclonal antibodies and IL-15 (n=9). (B) Mean and SEM (standard error of the mean) of the percentage of NK cells under proliferation among total NK cells in total (•), monocyte-depleted (∘) or monocyte/CD3⁺-depleted samples (*) after 7 days of culture (n=9) in the presence or not of monoclonal antibodies and IL-15.

FIG. 5: B leukemic cell depletion induced by monoclonal antibodies and IL-15 in CLL samples. Total, monocyte- or monocyte/CD3⁺-depleted samples from CLL patients were treated or not with RTX (10 μg/mL) or GA101 (10 μg/mL) with or without IL-15 (10 ng/mL) for 7 days then analyzed by flow cytometry. Percentage of CD19⁺/CD5⁺ viable cells compared to untreated cells after 7 days of treatment was assessed as described in Materials and Methods. (A) Absolute number of CD19⁺/CD5⁺ viable cells after 7 days of treatment (n=35) in total samples. Horizontal bars represent the median value (***p<0.001). (B) Mean and SEM (standard error of the mean) of the absolute number of CD19⁺/CD5⁺ viable cells after 7 days of treatment in total (•), monocyte-depleted (∘) or monocyte/CD⁺-depleted samples (*) (n=7).

FIG. 6: B leukemic cell depletion induced by monoclonal antibodies and IL-15 in CLL samples. PBMC from CLL patients were treated or not with RTX (10 μg/mL), GA101 (10 μg/mL), RTX F(ab′)₂ (10 μg/mL), GA101 F(ab′)₂ (10 μg/mL), during 7 days, then analyzed by flow cytometry. Percentage of CD19⁺/CD5⁺ viable cells compared to untreated cells after 7 days of treatment was assessed as described in Materials and Methods (n=7). Horizontal bars represent the median value (*p<0.05; **p<0.01).

### EXAMPLES

### Materials and Methods

### Cells and Reagents

Peripheral blood samples from untreated CLL patients (n=34, Table 1) were obtained with informed consent and referenced at the HIMIP laboratory. According to French law, HIMIP has been declared to the Ministry of Higher Education and Research (DC 2008-307 collection 1) and has obtained a transfer agreement (AC 2008-129) after approbation by an ethical committee (Comité de Protection des Personnes Sud-Ouest et Outremer II). Clinical and biological annotations of the samples have been declared to the CNIL (Comité National Informatique et Libertés: the Data Processing and Liberties National Committee).

Following separation by Ficoll gradient centrifugation, peripheral blood mononuclear cells (PBMC) were used immediately.

B leukemic cells were purified by magnetic separation without CD43 depletion using an EasySep® Human B Cell Enrichment Kit according to the manufacturer's instructions (Stemcell Technologies, Grenoble, France). NK cells from healthy donors were isolated from fresh buffy coats (obtained from Etablissement Francais du Sang, Toulouse, France) and purified using an EasySep® Human NK Cell Enrichment Kit (Stemcell Technologies, Grenoble, France). The purity of B leukemic cells or NK cells was assessed by flow cytometry and was between 90 and 98%.

Monocytes and T lymphocytes were depleted from whole blood samples using the RosetteSep® Human Monocyte Depletion Cocktail (CD36) and the RosetteSep® Human CD3 depletion cocktail respectively, according to the manufacturer's instructions (Stemcell Technologies, Grenoble, France). Depletion of Monocytes and T lymphocytes was assessed by flow cytometry and the level of remaining cells was below 0.1%.

RTX and GA101 monoclonal antibodies and RTX F(ab′)₂ and GA101 F(ab′)₂ fragments were provided by Roche Pharma (Basel, Switzerland). Recombinant Human IL-15 was purchased from Peprotech (Tebu-bio, Le Perray-en-Yvelines, France) and used at a final concentration of 10 ng/mL, as in previous studies.^{11,26,27}

For all experiments cells were cultured at 37° C. and 5% CO2 in RPMI supplemented with 10% heat-inactivated fetal calf serum (FCS), 2 mM L-glutamine, 100 μg/mL penicillin and 100 μg/mL streptomycin (Invitrogen, Cergy Pontoise, France). To provide long term viability CLL cultures were performed at high cell density (10×10⁶ cells/mL).

### Flow Cytometry

Monoclonal antibodies used for cell staining were: FITC-anti-CD3, Pacific Blue-anti-CD3, and PE-Cy-7-anti-CD5 (eBioscience, Paris, France); Pacific Blue-anti-CD19 and PE-Cy-7-anti-CD16 from BioLegend (Ozyme, Saint-Quentin-en-Yvelines, France); PE-anti-CD69 and PE-Cy7-anti-CD56 (Beckman-Coulter, Roissy, France); and isotype-matched control conjugates. Briefly, PBMC or purified cells were washed with cold PBS containing 1% FCS, stained with the appropriate conjugated antibodies on ice for 30 min, then washed and analyzed using a BD LSR II cytometer (BD Biosciences, Pont de Claix, France) and DIVA software.

NK Cell Activation Assays from CLL Samples

Fresh PBMC from untreated CLL patients were seeded at 10×10⁶ cells/mL in culture medium and were either left untreated or treated with RTX (10 μg/mL) or GA101 (10 μg/mL) for 7 days. When appropriate, recombinant human IL-15 was added at a final concentration of 10 ng/mL. Activation of NK cells was evaluated by flow cytometry detecting CD69 expression on CD3⁻/CD56⁺ gated cells.

### NK Cell Proliferation Assay

Freshly-isolated PBMC from CLL patients or purified NK cells from healthy donors (5×10⁶ cells/mL) were labeled with 1 μM CFSE (Carboxyfluorescein diacetate succinimidyl ester) (Invitrogen/Molecular Probes, Carlsbad, Calif.) for 10 min at 37° C. and washed with PBS according to the manufacturer's instructions. Labeled cells were then cultivated in complete medium with or without RTX (10 μg/mL) or GA101 (10 μg/mL), and/or 10 ng/mL IL-15, for 7 days. For some experiments, CFSE-labeled purified NK cells from healthy donors were incubated in 100% B leukemic cell supernatant supplemented or not with RTX (10 μg/mL) or GA101 (10 μg/mL). In all experiments CFSE dilution was analyzed on CD3⁻/CD56⁺ gated cells by flow cytometry.

### Production of CLL Supernatants

Purified B leukemic cells were seeded at 10×10⁶ cells/mL in culture medium and incubated with or without IL-15 (10 ng/mL). After 7 days cells were centrifuged. Supernatants were filtered and used immediately or stored at −80° C.

In Vitro B Leukemic Cell Depletion Assays from CLL Samples

Fresh PBMC from untreated CLL patients were seeded at 10×10⁶ cells/mL in culture medium and were left either untreated or treated with RTX (10 μg/mL) or GA101 (10 μg/mL) for 7 days. When appropriate, IL-15 was added at a final concentration of 10 ng/mL. B leukemic cell depletion was based on total viable cell number determination (by trypan blue exclusion) combined with the percentage of viable CD19⁺/CD5⁺ lymphocytes determined by flow cytometry and was calculated as follows:

CD19⁺/CD5⁺ absolute number of viable cells: Viable cell number determination x % CD19⁺CD5⁺

% of CD19⁺/CD5⁺viable cells=100×(Absolute number in treated samples/Absolute number in untreated samples).

### Autologous Chromium-Release Cytotoxic Assay

Natural cytotoxicity of effector cells from 4 random CLL patients was tested using the classical chromium release assay. Briefly, NK cells were purified from PBL samples using a custom RosetteSep® Human NK Cell Enrichment Kit (#R17523, Stemcell Technologies, Grenoble, France) and stimulated or not with IL-15 (10 ng/mL). Autologous B leukemic cells (target cells) were incubated in RPMI 1% FCS for 1 h at 37° C. with ⁵¹Cr (Sodium Chromate, Perkin Elmer, Courtaboeuf, France) (100 μCi per 10⁶ cells). Cells were then washed and plated at 10⁴/well in round-bottom 96-well plates. Increasing amounts of effector cells were added to triplicate wells with an NK/target ratio ranging from 0.01/1 to 0.8/1. Control wells contained only target cells to measure spontaneous release or target cells with 0.1% Triton X-100 to measure maximal release. After centrifugation, plates were incubated for 4 hrs at 37° C., 5% CO2. 50 μL were then collected from each well and counted in a gamma-counter. Percentage of specific lysis was calculated as follows: ((sample release−spontaneous release)/(maximal release−spontaneous release))×100.

### Quantification of CD20 Expression on B Leukemic Cells

CD20 expression was quantified using the BD QuantiBRITE fluorescent assay (BD Biosciences, Le Pont de Claix, France) on CD19⁺/CD5⁺ gated cells by flow cytometry. The antibody bound per cell (ABC) value represents the mean value of the maximum capacity of each cell to bind the anti-CD20 antibody and was evaluated according to the manufacturer's instructions.

### Statistics

Paired or unpaired Student's t-tests were used to determine differences between samples as appropriate. P values lower than 0.05 were considered statistically significant.

### Results

### NK Activation Induced by Monoclonal Antibodies and IL-15 in CLL Samples

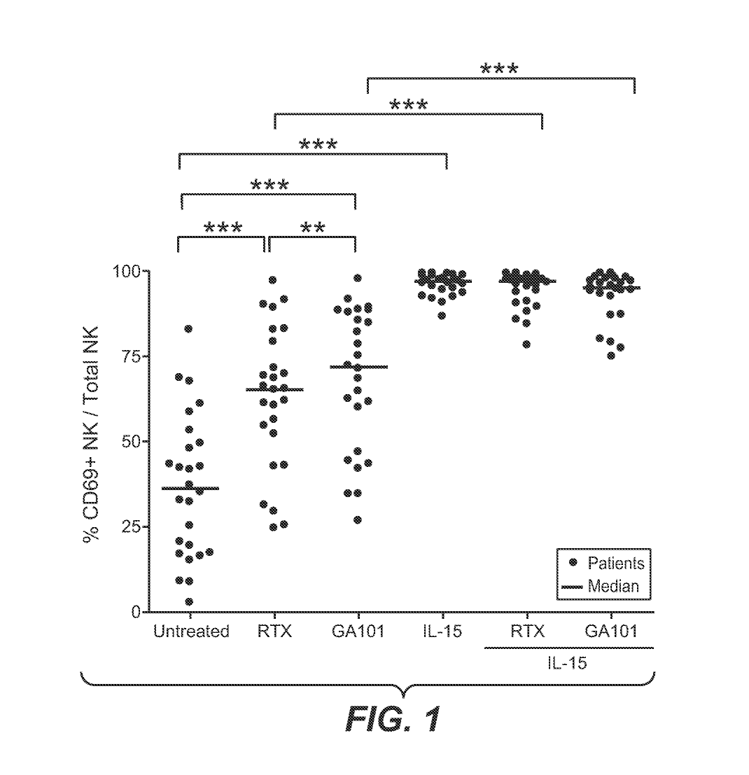

NK activation was evaluated in random CLL samples (n=26) using CD69 expression as a cell-surface activation marker (FIG. 1). CD69 is an early activation marker whose expression is sustained during culture (data not shown). In untreated samples NK activation was observed only after 7 days of culture. Incubation with RTX or GA101 led to an increase in activated NK cells (% of CD69⁺ cells among total NK cells had a median of 65.25% and 72.25% for RTX and GA101 respectively) when compared with untreated cells (median: 35.75%). It is noteworthy that this phenomenon was significantly higher with GA101 than RTX (p<0.01). The observed NK cell activation was dependent upon the Fc portion of monoclonal antibodies as no significant activation was observed using F(ab′)₂ fragments (data not shown), highlighting the importance of CD16 signaling (via antibody-Fc-fragment binding to its receptor). Addition of IL-15 led to activation of all NK cells (median: 95.25%), without synergistic or additive effects induced by RTX or GA101 (median: 96.75% and 95.75% respectively).

### NK Proliferation Induced by Monoclonal Antibodies and IL-15 in CLL Samples

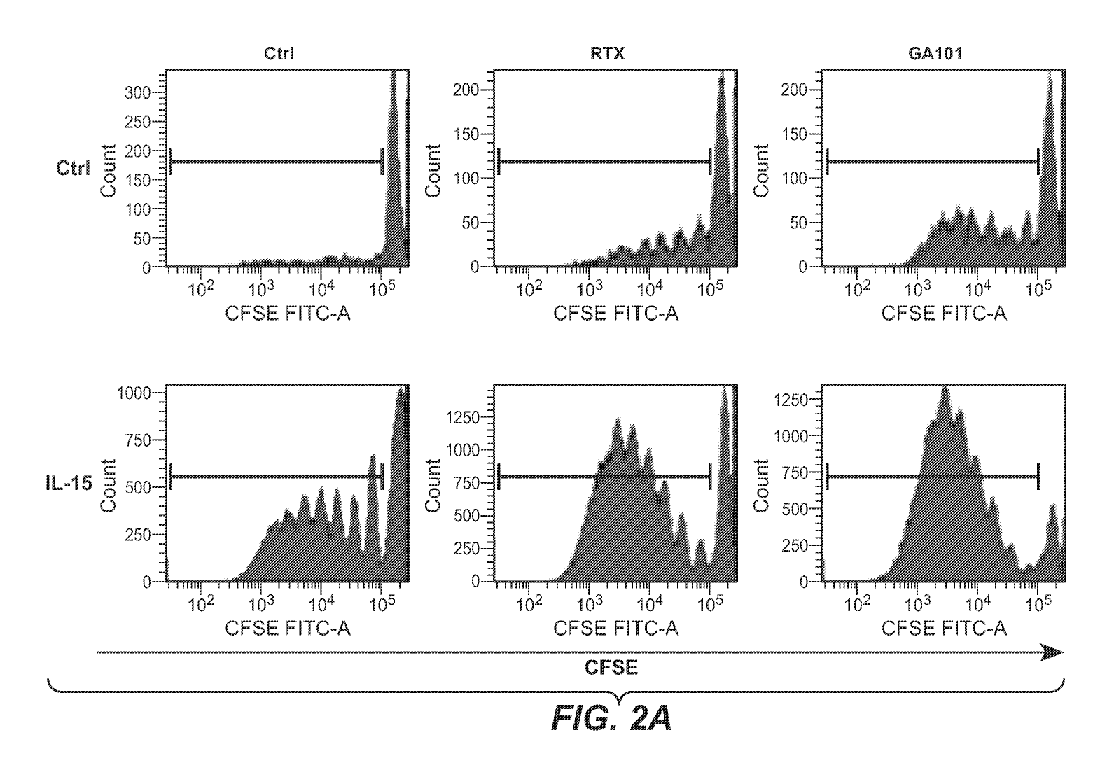

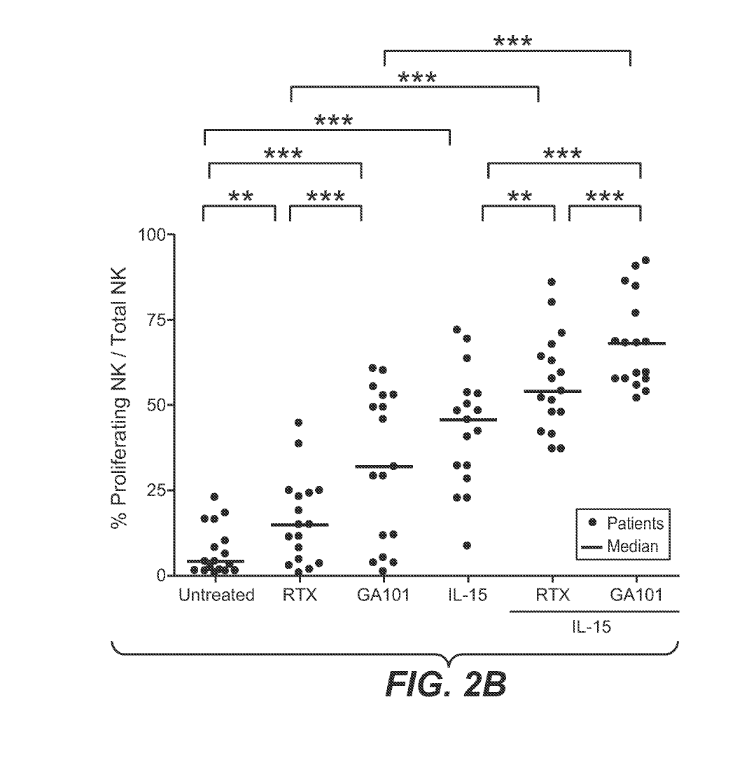

The combined effect of IL-15 and monoclonal antibodies on NK cell proliferation was evaluated in 7-day CLL cultures. Results show a proliferation of gated CD3⁻/CD56⁺ cells induced by RTX and GA101 alone, characterized by CFSE dilution, with a significantly higher proportion of proliferating NK cells in GA101-treated than RTX-treated samples (median % of proliferating NK cells: 31.75% vs 14.75% respectively) (FIG. 2). As observed for NK activation, these results confirm the importance of CD 16 signaling in NK-cell proliferation. In addition, IL-15 treatment induced a strong proliferation of NK cells (median: 42.25%). Importantly, this phenomenon was significantly greater with the combination of IL-15 plus monoclonal antibodies, with a stronger NK proliferation induced by IL-15/GA101 compared to IL-15/RTX (median: 67.25% vs 53.75% respectively; p<0.001). This effect was not due to an increase in CD16 expression in NK cells incubated with IL-15 (the mean fluorescence intensity of CD16 expression in samples incubated with IL-15 was 5481±3395 vs 6491±2644 in medium alone; p=0.102). These data support the hypothesis that cooperation of IL-15 and CD16 signaling is important in NK cell proliferation.

### No Proliferation of Purified NK Cells Induced by IL-15 and Monoclonal Antibodies

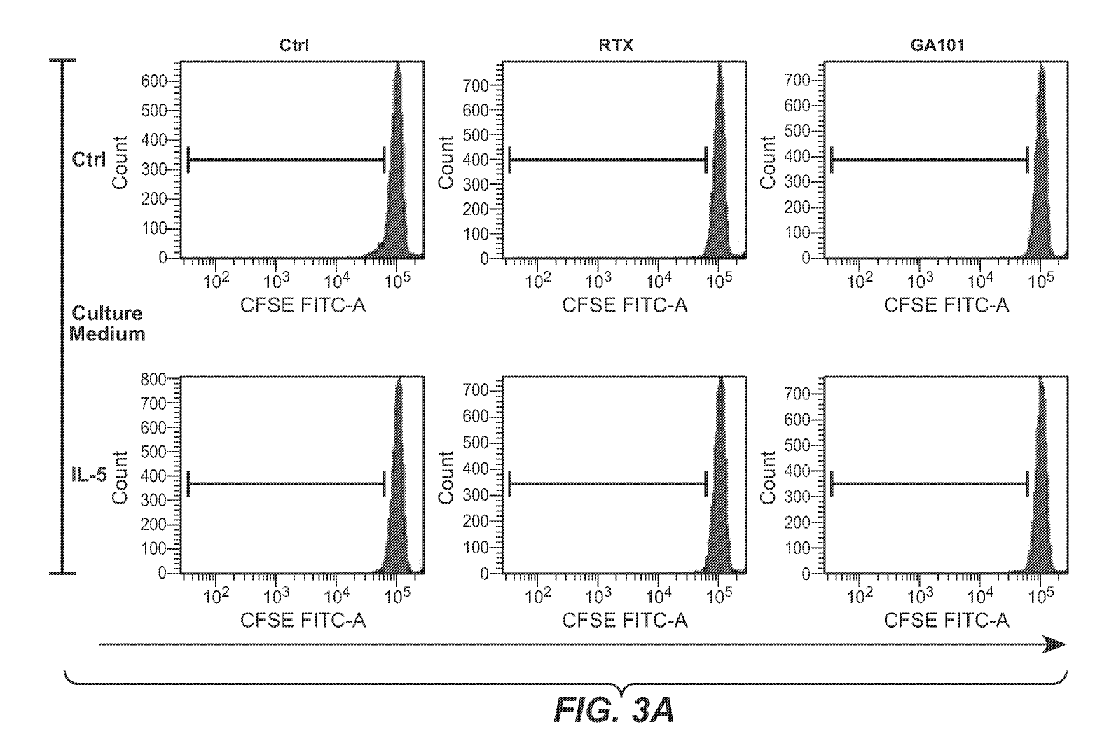

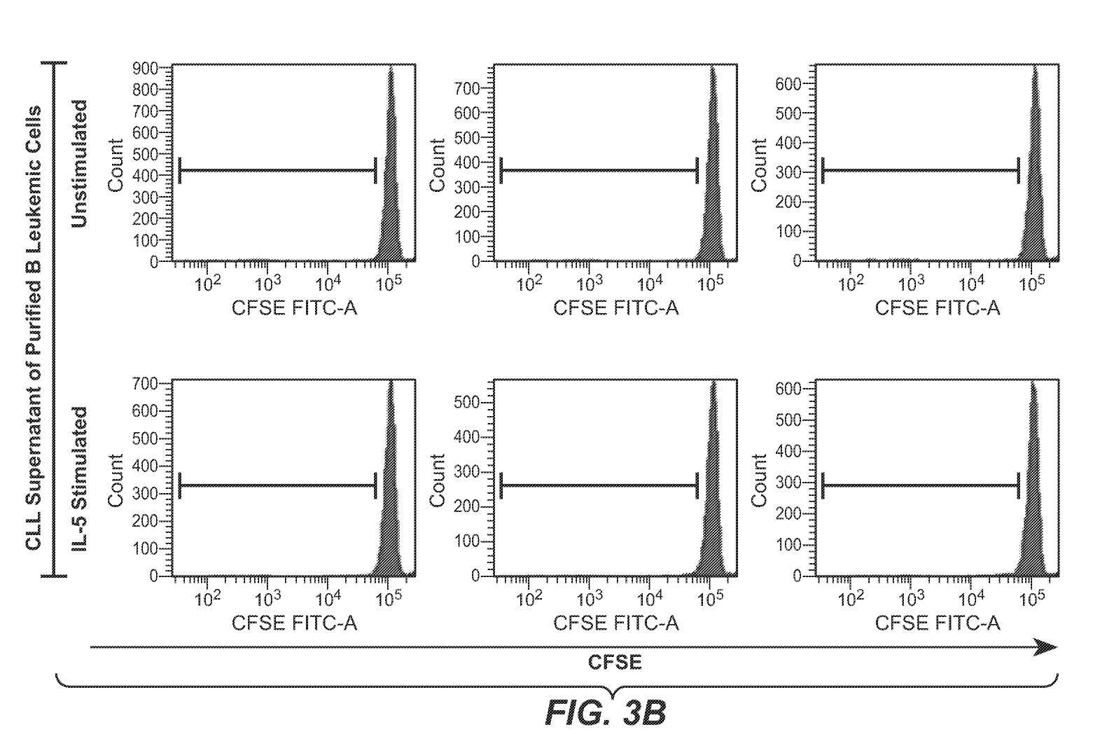

CLL cells have the capacity to release soluble factors which have paracrine or autocrine activities and are thus able to regulate immune effector functions.¹⁴ To evaluate whether B-CLL-released soluble factors can affect NK proliferation, purified NK cells from healthy donors were cultured for 7 days in appropriate medium or in IL-15-stimulated CLL cell supernatants with or without monoclonal antibodies. Culture at high density for 7 days can lead to a decrease of medium nutrients and impact cell viability. In our experiments the absolute number of NK viable cells cultured in medium or B-CLL supernatants is identical under both conditions (p=0.45). As shown in FIG. 3, no NK proliferation was observed either in culture medium alone or in the presence of IL-15 with or without monoclonal antibodies. Addition of B-CLL supernatants from either untreated or IL-15-stimulated purified B leukemic cells did not induce better NK proliferation in the presence or absence of monoclonal antibodies, suggesting that B-CLL soluble factors were not involved in NK proliferation and supporting the hypothesis of a cell-cell interaction for this process.

### IL-15 Trans Presentation by CLL-Cells Induced NK Proliferation by Monoclonal Antibodies

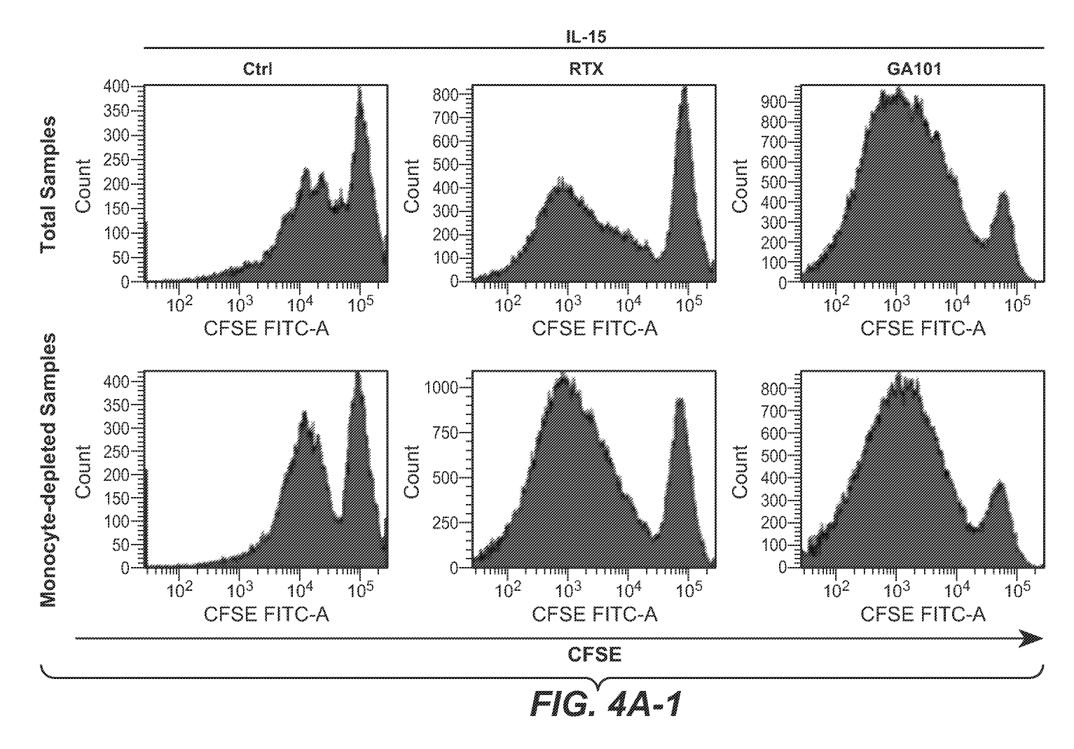

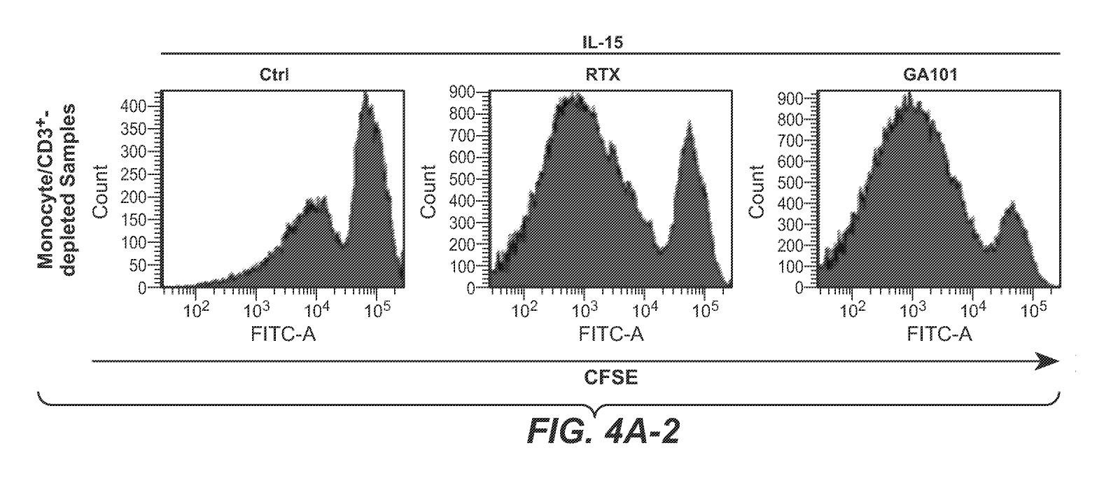

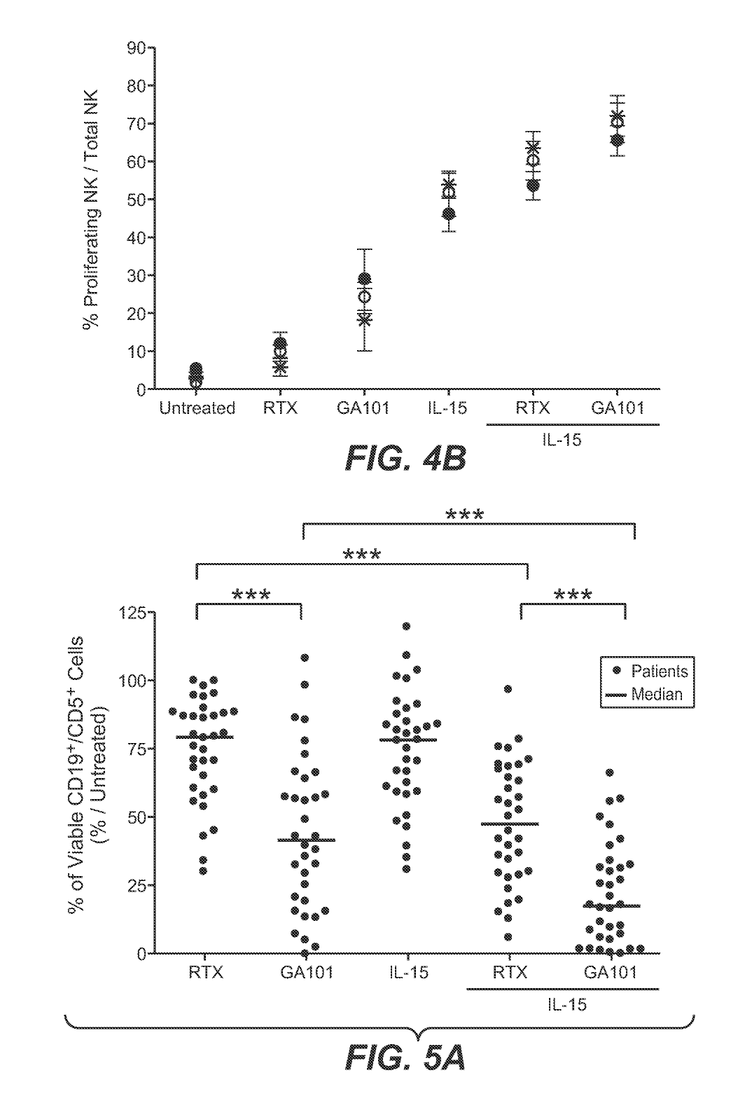

As previously described, the prevailing mechanism of IL-15 action is trans-presentation by accessory cells such as monocytes. Despite the relatively low number of monocytes in CLL samples due to B cell accumulation (Table 1) we explored the contribution of IL-15 trans-presentation in NK proliferation using successive depletion of IL-15Ra-positive cells. Results show that, compared to total samples, NK proliferation was unchanged in both monocyte- and monocyte/CD3⁺-depleted CLL samples (p=0.602 and p=0.775 respectively) (FIG. 4). These data confirm the hypothesis that an interaction between B-leukemic and NK cells is important to stimulate NK proliferation in the presence of IL-15. The improved proliferation observed in the presence of monoclonal antibodies could be attributed to a stronger interaction between B and NK cells via CD20 recognition. It is noteworthy that IL-15 did not modulate CD20 expression on B leukemic cells (p=0.36). Altogether these results strongly demonstrate that B leukemic cells act as accessory cells for IL-15 trans-presentation to NK cells.

### Co-Activity of IL-15 and Monoclonal Antibodies in B Leukemic Cell Depletion

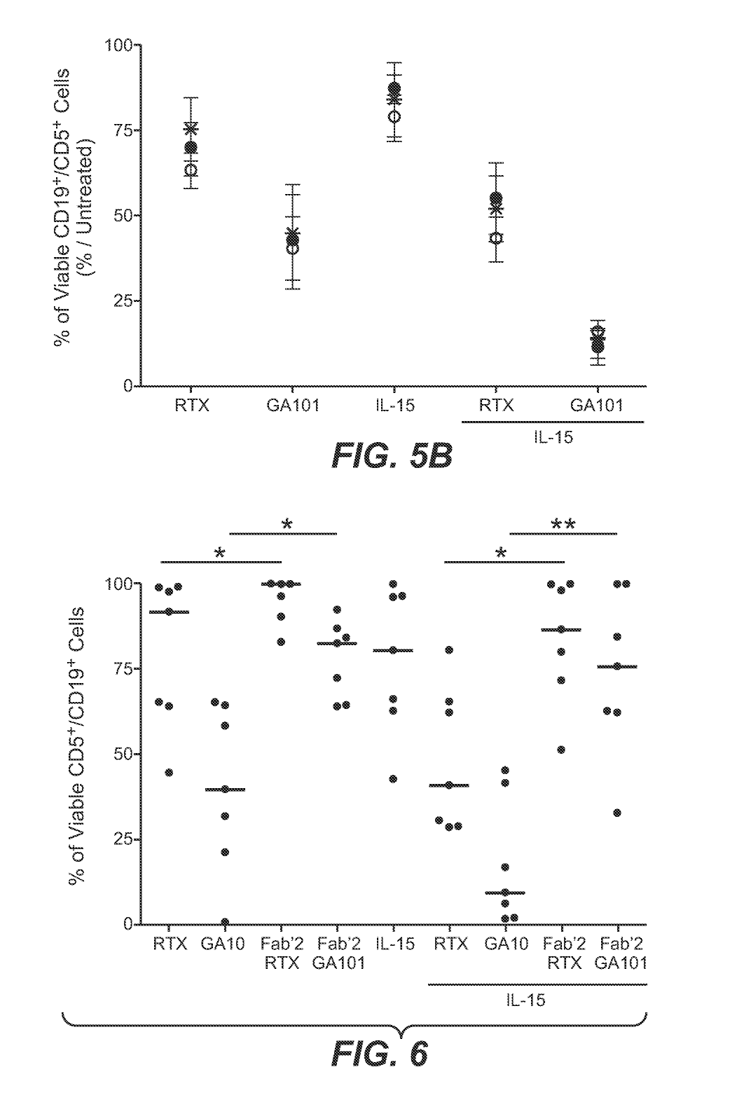

The functional implication of IL-15-stimulated NK cells was assessed by B leukemic cell depletion assays in CLL samples from untreated patients (n=34, Table 1). B-cell cultures were performed at high cell density (10×10⁶ cells/mL) to provide longevity, as they spontaneously die at low density.^{13,29} At this high cell concentration, spontaneous CLL death did not exceed 34% after 7 days. Surprisingly, the results show that at first IL-15 alone induced a slight B cell depletion (FIG. 5A). Thus we tested the effect of IL-15 stimulation on NK cytotoxic function against autologous B leukemic cells. At a 0.02/1 NK/B ratio (relevant to the NK/B ratio observed in B-CLL samples, Table 1) IL-15 was able to increase the natural cytotoxicity of NK cells (% of specific cytotoxicity: 0.03±0.1% for NK cells; 8.9±1.9% for IL-15-stimulated-NK cells; p=0.004).

In PBMC whole samples, GA101 displayed a greater cytotoxic activity than RTX in terms of CD19⁺/CD5⁺ cell depletion (median of viable B leukemic cells 39.9% vs 79.5% respectively; p<0.0001) (FIG. 5A). This observed B cell depletion was dependent upon the Fc portion of monoclonal antibodies since no significant depletion was observed using F(ab′)₂ fragments (FIG. 6) and was not related to CDC due to culture conditions. Most importantly, RTX- or GA101-mediated B-cell depletion was significantly increased in the presence of IL-15 (median of viable B leukemic cells: RTX 79.5% vs RTX/IL-15 50.4%, p<0.0001; GA101 39.9% vs GA101/IL-15 17.8%, p<0.0006) (FIG. 5A). These observations suggest a cooperative role of IL-15 and CD16 activation in RTX- or GA101-mediated leukemic cell depletion in CLL samples and highlight a greater efficacy of the combination of IL-15/GA101 in this process.

To confirm that depletion of B leukemic cells was strictly NK-dependent, monocytes and T-lymphocytes were successively removed from CLL samples. Results show that the removal of monocytes, as well as both monocytes/T-lymphocytes, does not affect B cell depletion induced by RTX or GA101 with or without IL-15 (compared to total samples, p>0.51 for monocyte-depleted samples; p>0.59 for monocyte/T-lymphocyte-depleted samples) (FIG. 5B).

Altogether, these data highlight the strong effect of IL-15 on NK cells in CLL leading to an increase in both natural autologous cytotoxicity and ADCC.

What is claimed:

1. Anti-CD20 antibody in combination with human IL-15 for use in treatment of cancer.

2. Anti-CD20 antibody according to claim 1 characterized in that said CD20 antibody is an afucosylated antibody with an amount of fucose of 60% or less, and said cancer is a CD20 expressing cancer.

3. Anti-CD20 antibody according to any one of claim 2, characterized in that said anti-CD20 antibody is a humanized B-Ly1 antibody.

4. Anti-CD20 antibody according to any one of claims 1 to 3, characterized in that said anti-CD20 antibody is obinutuzumab.

5. Anti-CD20 antibody according to any one of claims 1 to 4, characterized in that said the cancer is leukemia.

6. Anti-CD20 antibody according to any one of claims 1 to 5, characterized in that said the cancer is chronic lymphocytic leukemia.

7. Anti-CD20 antibody according to any one of claims 1 to 6, characterized in that the Anti-CD20 antibody is an afucosylated antibody which shows an increased ADCC.

8. Anti-CD20 antibody according to any one of claims 1 to 7, characterized in that one or more additional other cytotoxic, chemotherapeutic or anti-cancer agents, or compounds or ionizing radiation that enhance the effects of such agents are administered.

9. A method for the treatment of cancer, comprising administering to a patient in need of such treatment an anti-CD20 antibody and a human IL-15.

10. The method of claim 9, comprising co-administering the anti-CD20 antibody and the human IL-15.

11. The method of claim 9 or 10, wherein the anti-CD20 antibody and the human IL-15 are administered sequentially or simultaneously.

12. The method according to claim 9, wherein the CD20 antibody is an afucosylated antibody with an amount of fucose of 60% or less, and said cancer is a CD20 expressing cancer.

13. The method according to claim 9, wherein the anti-CD20 antibody is a humanized B-Ly1 antibody.

14. The method according to claim 9, wherein the anti-CD20 antibody is obinutuzumab.

15. The method according to claim 9, wherein the cancer is leukemia.

16. The method according to claim 15, wherein the leukemia is chronic lymphocytic leukemia.

17. The method according to claim 9, wherein the anti-CD20 antibody is an afucosylated antibody which shows an increased ADCC.

18. The method according to claim 9, wherein one or more additional other cytotoxic agent, chemotherapeutic agent, anti-cancer agent, ionizing radiation, compound, or agents that enhance the effects of such agents are administered.

19. A composition comprising an anti-CD20 antibody and a human IL-15.

20. The composition according to claim 19, further comprising a cytotoxic agent, chemotherapeutic agent or an anti-cancer agent.
